(19) United States
(12) Patent Application Publication
(10) Pub. No.: US 2012/0045525 A1
(43) Pub. Date: Feb. 23, 2012

(54) MOSQUITO REPELLENT SOLUTION, PREPARATION METHOD AND USE THEREOF

(75) Inventor: Jianyi Ma, Zhejiang (CN)

(73) Assignee: ZHEJIANG FORESTRY COLLEGE, Linan, Zhejiang (CN)

(21) Appl. No.: 13/145,010

(22) PCT Filed: Nov. 16, 2009
(86) PCT No.: PCT/CN09/01262

§ 371 Date: Jul. 18, 2011

(30) Foreign Application Priority Data
Nov. 20, 2008 (CN) 200810162516.0
May 21, 2009 (CN) 200910098862.1

(51) Int. Cl. A01N 37/02; A61P 31/00; A01P 17/00; A61P 17/00; A01N 37/04; A61K 33/14

(52) U.S. Cl. 424/680; 514/557; 514/574

(57) ABSTRACT

A mosquito repellent solution to prevent mosquito bite, comprising 0.1-9.9% organic acid of total weight as active principle, 88-99.6% water as solvent. The organic acid is single acid or mixed composition out of ethyl butyrate, propionic acid, n-butyric acid, isobutyric acid, isobutyric, valeric acid, hexanoic acid, n-Heptanoic acid, L-Lactic Acid and malic acid. The composition solution to prevent mosquito bite disturb mosquitoes' olfactory sensation through imitating human's smell of skin and breath; all raw materials base on the standard of food grade, so the composition is safe to human beings, particularly to infant, pregnant women, children and the old who are weaker. The composition solution does not work by killing mosquitoes so it would not relate to resistance and ecology risk. The preparation method is simple with low costs; there is no three wastes and friendly to the environment. The composition solution applies to the preparation of preventing mosquito bite, relieving itching and detumescence, and air sterilization.

### FIELD OF THE INVENTION

The present patent relates to Household chemical field and more particularly to the preparation method and the use of a mosquito repellent solution as a smell interference agent to female mosquito.

### BACKGROUND OF THE INVENTION

Mosquito would make people feel itchy and transmit diseases by biting humans for blood. Generally, only female mosquito bites people, not the male. It is showed that mosquitoes find their target by certain smell in long distance; this smell is human's sweat. The analysis and research about sweat show that mosquitoes find the position of their target by organic acids in sweat, for their olfactory organs have organic acid receptor protein; those organic acids contain lactic acid which can stimulate mosquitoes feeling exciting. While short chain organic acid with high concentration, such as ethyl butyrate, propionic acid and butyric acid, would restrain mosquitoes' excitement, which would make the mosquitoes feel not like to find the target to bite; short chain organic acid with low concentration works on the contrary way, the mosquitoes would be very exciting. If some certain environment is full of these low concentration organic acid, mosquitoes would be over exciting and have wrong feeling that there were full of people in the room, thus covered human's lactic acid in sweat and disturbed mosquitoes' Olfactory functions to find the right target.

Early research showed that mosquitoes found target by Carbon dioxide breathed out of target. But the following research proved it is misunderstand; mosquitoes cannot find target by carbon dioxide. The present study show that female mosquitoes find their target by other smell (the smell of half-digested food in stomach instead of carbon dioxide). besides, mosquitoes can also find target by temperature and humanity of human bodies.

The active principles in Traditional mosquito-repellent incense are pesticide, particularly Carbamate and pyrethroid insecticides which belong to cholinesterase (AChE) inhibitors and sodium channel regulator and take effect in animals' nerve system as neurotoxin. But the method taking effect by killing mosquitoes was also very harmful to human beings with its long-term use. As one part of Ecological chain, excessive killing mosquitoes may have negative effect on nature. And mosquitoes may have resistance to widely used Cyhalothrin pesticide because of their strong reproduction ability. So the best way to prevent mosquitoes bite while considering the ecology is to disturb their olfactory functions instead of killing them.

So far, there are also some products containing the information of human sweat in the market, such as N,N-diethyl-3-methylbenzamide (DEET), it volatilizes easily, and works by block mosquitoes' 1-octene-3-alcoho as olfactory receptor; but the pests just loss their feeling to human or animal's certain smell, their olfactory ability to human breath is as usual. And recent study showed that DEET as a repellent works through making pests feeling wrong, thus to decrease the chances being bitted, and the effect is poor. The pesticide itself also has toxin and may do harm to human.

### SUMMARY OF THE INVENTION

Addressing the foregoing problems affecting the existing technology, the goal of the present invention is to find a Technical method to get a mosquito repellent solution preventing mosquito bite, its preparation method and the use.

The pesticide solution should be with good efficacy and provide a simple and low-costing preparation method thereof and no harm to human, also no contamination while in producing period.

The claimed mosquito repellent solution comprising: 0.1-9.9% organic acid of total weight as active ingredient, 88-99.6% water of total weight as solvent, the organic acid comprises at least one organic acid selected from the group consisting of acetic acid, propanoic acid, n-butyric acid, isobutyric acid, isobutyric acid, valeric acid, hexanoic acid, n-Heptanoic acid, L-Lactic acid and malic acid.

The claimed mosquito repellent solution, wherein the organic acid is 1.0-8.0% by weight of the mosquito repellent solution, it is better that the organic acid is 2.0-6.0% by weight of the mosquito repellent solution.

The claimed mosquito repellent solution, the water as solvent is distilled water, the distilled water has 90-98% by weight of the mosquito repellent solution, it is better that the distilled water has 93-96% by weight of the mosquito repellent solution.

The claimed mosquito repellent solution, the organic acid is in a food level.

The claimed mosquito repellent solution, wherein 0.1-0.5% sodium chloride or citric acid of total weight is used as antiseptic.

The claimed mosquito repellent solution, wherein 0.1-0.5% water-soluble fruit flavor spices of total weight is added as sauce.

The claimed a preparation method for the mosquito repellent solution comprising following steps:
place a right proportion of distilled water and organic acid in a heating mixer, heat the mixture of distilled water and organic acid to 15-35° C. while constantly stirring the mixture to promote dissolve, then yield the final product of the mosquito repellent solution.

The claimed preparation method, wherein antiseptics and sauce are added in order during constantly stirring to promote dissolve.

The claimed preparation method, wherein the mixture is heated to 20-30° C., better to 20-25° C.

The claimed a use of the mosquito repellent solution comprising preventing mosquito bite, relieving itching, detumescence and air sterilization.

Portions and percents in this patent application are all unit of weight unless there is other explanation. And related equipment and raw material are easily purchased from the market, or frequently-used for this industry.

### DETAIL DESCRIPTION OF THE INVENTION

The examples summarized below provide a concrete explanation of the contents of this invention.

### Preparation Example 1

Add 90 kg distilled water to a reaction vessel; slowly add 9.9 kg acetic acid (with purity over 99%.) of food level. Heat to 20° C. while constantly stirring to promote dissolving fully, add 0.1 kg citric acid, stir thoroughly, yielding the final product—a mosquito repellent solution to prevent mosquito bite.

### Preparation Example 2

Add 99.6 kg distilled water to a reaction vessel; slowly add 0.1 kg propanoic acid with a purity of 99.8% of food level. Heat to 15° C. while constantly stirring to promote dissolving fully, add 0.2 kg Sodium chloride, stir thoroughly, add 0.1 kg water-soluble apple flavor spices, stir to promote dissolving fully, yielding the final product—a mosquito repellent solution to prevent mosquito bite.

### Preparation Example 3

Add 98.5 kg distilled water to a 100 L reaction vessel; slowly add 0.5 kg acetic acid and 0.5 kg malic acid both of food level. Heat to 25° C. while constantly stirring to promote dissolving fully, add 0.3 kg Sodium chloride, stir thoroughly, add 0.2 kg water-soluble lemon flavor spices, stir to promote dissolving fully, yielding the final product—a mosquito repellent solution to prevent mosquito bite.

### Preparation Example 4

Add 91.3 kg distilled water to a 100 L reaction vessel; slowly add 6.0 kg propionic acid, 0.5 kg acetic acid, 0.5 kg n-Butyric acid; 0.5 kg isobutyric acid, 0.5 kg valeric acid, heat to 25° C. while constantly stirring to promote dissolving fully, add 0.4 kg Sodium chloride, stir thoroughly, add 0.3 kg water-soluble lemon flavor spices, stir to promote dissolving fully, yielding the final product—a mosquito repellent solution to prevent mosquito bite.

### Preparation Example 5

Add 97.1 kg distilled water to a 100 L reaction vessel; slowly add 0.9 kg z n-Butyric acid; 0.9 kg isobutyric acid, 0.1 kg hexanoic acid, 0.1 kg heptylic acid, heat to 30° C. while constantly stirring to promote dissolving fully, add 0.5 kg Sodium chloride, stir thoroughly, add 0.4 kg water-soluble orange flavor spices, stir to promote dissolving fully, yielding the final product—a mosquito repellent solution to prevent mosquito bite.

### Preparation Example 6

Add 93 kg distilled water to a 100 L reaction vessel, slowly add 2 kg valeric acid, 2 kg Hexanoic acid, 2 kg heptylic acid, heat to 35° C. while constantly stirring to promote dissolving fully, add 0.5 kg Citric acid, stir thoroughly, add 0.5 kg water-soluble watermelon flavor spices, stir to promote dissolving fully, yielding the final product—a mosquito repellent solution to prevent mosquito bite.

### Preparation Example 7

Add 95 kg distilled water to a 100 L reaction vessel, slowly add 3 kg acetic acid, 0.2 kg propionic acid. 0.2 kg n-Butyric acid. 0.2 kg isobutyric acid, 0.2 kg valeric acid, 0.2 kg hexanoic acid. 0.2 kg n-heptylic acid, 0.2 kg L-lactic acid, 0.6 kg malic acid, heat to 25° C. while constantly stirring to promote dissolving fully yielding the final product—a mosquito repellent solution to prevent mosquito bite.

The mosquito repellent solution to prevent mosquito bite is safe to human beings. In order to improve its safety to human, Synthetic chemicals such as spice should be used as less as possible. Considering this composition solution is liquid, and used by volatilizing into gas, antiseptic Sodium chloride should be less used or even no use to decrease solid residual and reduce the block of water channels by solid while in volatilization process.

The below Composition test results for preparation examples would show its efficacy

Test 1: test method base on GB13917.8—1992, testing the mosquitos' density in the field by human body calling. Arrange 2 investigators in every test room (investigators, place and time should be fixed), every tester roll up the trouser legs to knee to reveal the skin, lighting constantly with a flashlight, catch mosquitoes fall on the naked legs with motor-driven mosquito dispeller for 45 minutes, then identify the mosquitoes' quantity, gender and type. Mosquito density decent rate (%)=1-mosquito density of test room/mosquito density of blank control room.

TABLE 1
mosquito density and the decrease rate after using Preparation Example 1-7

| | | | | | | The |
|---|---|---|---|---|---|---|
| | | | | | | decrease |
| | | | | | | rate of |
| | Quantity of caught mosquitoes | | | | | density |
| preparation example | room1 | room2 | room3 | room4 | average | (%) |
| preparation example 1 | 2 | 2 | 3 | 3 | 2.5 (1.58-3.42) | 93.33 |
| preparation example 2 | 5 | 5 | 6 | 5 | 5.25 (4.45-6.05) | 86.00 |
| preparation example 3 | 4 | 6 | 5 | 7 | 5.5 (3.45-7.55) | 85.33 |
| preparation example 4 | 5 | 6 | 7 | 5 | 5.75 (4.23-7.28) | 84.67 |
| preparation example 5 | 4 | 5 | 5 | 6 | 5 (3.70-6.30) | 86.67 |
| preparation example 6 | 6 | 6 | 7 | 8 | 6.75 (5.23-8.27) | 82.00 |
| preparation example 7 | 2 | 3 | 5 | 4 | 3.5 (1.45-5.55) | 90.67 |
| Liquid | 7 | 6 | 7 | 7 | 6.75 (5.95-7.55) | 82.00 |
| mosquitoes-repellent | | | | | | |
| incense | | | | | | |
| | 28 | 34 | 43 | 45 | 37.5 (24.87-50.13) | |

Table 1 showed that mosquito density decreased significantly after using the mosquito repellent solution Preparation Example 1-7, the decrease rates of mosquito density all are over 80%. the rates in Preparation Example 1 and Preparation Example 7 are over 90%. Preparation example 2-6 reaches the effect of traditional mosquito-repellent incense, while preparation example 1 and preparation example 7 are better than traditional ones.

Test 2: test method base on GB13917.8—92, testing the mosquitos' density in laboratory room with outside strain mosquitoes. According to GB13917.4-92, Culex pipiens quinquefasciatus, collected in the field, are used to test the products made from preparation example 1-7 after breeding and eclosion in the lab for 2 or 3 days; human body tempts the mosquitoes then observe the prevention efficacy. Mosquito density decrease rate (%)=1-mosquito density of test room/mosquito density of blank control room.

TABLE 2
the efficacy of preventing mosquito bite after using preparation
example 1-7

| type of | Times being bitten (time/2 h) | | | | |
|---|---|---|---|---|---|
| mosquitoes- | | | | Average (95% | Prevention |
| repellent | | | | confidence | efficacy |
| incense | repeat 1 | repeat 2 | repeat 3 | intervals) | (%) |
| Preparation | 0 | 0 | 0 | 0 (0-0) | 100 |
| example 1 | | | | | |
| Preparation | 1 | 2 | 2 | 1.67 (0.23-3.10) | 88.89 |
| example 2 | | | | | |
| Preparation | 1 | 1 | 3 | 1.67 (−1.20-4.53) | 88.89 |
| example 3 | | | | | |
| Preparation | 2 | 2 | 3 | 2.33 (0.89-3.76) | 84.44 |
| example 4 | | | | | |
| Preparation | 2 | 3 | 4 | 3 (0.51-5.48) | 80.00 |
| example 5 | | | | | |
| Preparation | 1 | 2 | 3 | 2 (−0.48-4.48) | 86.67 |
| example 6 | | | | | |
| Preparation | 0 | 2 | 0 | 0.67 (−2.20-3.53) | 95.56 |
| example 7 | | | | | |
| Liquid | 3 | 3 | 5 | 3.67 (0.80-6.54) | 75.53 |
| mosquitoes- | | | | | |
| repellent | | | | | |
| incense | | | | | |
| | 13 | 14 | 18 | 15 (8.42-21.57) | |

Table 2 showed that times of bitten by mosquitoes decreased significantly after using the mosquito repellent solution Preparation Example 1-7, protection efficacies of all compositions are over 80%. the protection efficacies in Preparation Example 1 and Preparation Example 7 are over 90%, they have much higher efficacy than traditional mosquito-repellent incense, especially the protection efficacy of preparation example 1 reaches 100%. Preparation example 2-6 reach the effect of traditional mosquito-repellent incense are just better than traditional ones.

Test 3: 20 testers are involved, everyone is bitten with 5 times, so total 100 person-times; testers should use mist spray made from the mosquito repellent solution of preparation example 1-7 immediately within 5 cm diameter after bitten by mosquitoes, then begin timekeeping, record the efficacy of relieving itching in 1 minute, 3 minute, efficacy of detumescence in 1 minute, 5 minutes, 10 minutes and 30 minutes. computing method: percentage of relieving itching=person-time of relieving itching total person-time×100; percentage of detumescence=person-time of detumescence/total person-time×100.

TABLE 3
relieving itching and detumescence after using the composition solution of preparation
example 1-7 (mist spray)

| | Percentage | Percentage | Percentage | Percentage | Percentage | |
|---|---|---|---|---|---|---|
| | of | of | of | of | of | Percentage of |
| | relieving | relieving | detumescence | detumescence | detumescence | detumescence |
| | in 1 min % | in 3 min % | in 1 min % | in 5 min % | in 10 min % | in 30 min |
| Preparation | 82 | 93 | 52 | 63 | 71 | 85 |
| example1 | | | | | | |
| Preparation | 76 | 87 | 45 | 60 | 65 | 74 |
| example 2 | | | | | | |
| Preparation | 74 | 84 | 43 | 55 | 68 | 76 |
| example 3 | | | | | | |
| Preparation | 70 | 80 | 41 | 61 | 69 | 75 |
| example 4 | | | | | | |
| Preparation | 65 | 76 | 38 | 55 | 62 | 69 |
| example 5 | | | | | | |
| Preparation | 64 | 77 | 41 | 55 | 60 | 71 |
| example 6 | | | | | | |
| Preparation | 66 | 77 | 43 | 59 | 64 | 76 |
| example 6 | | | | | | |
| Preparation | 78 | 90 | 48 | 61 | 69 | 81 |
| example 7 | | | | | | |

Table 3 showed that the efficacy of relieving itching reached 64-82% in 1 minute, 77-93% in 3 minutes with the mist spray made from preparation example 1-7, especially preparation example 1 and preparation example 7 have the better efficacy. The efficacy of detumescence reached 41-52% in 1 minute, 55-63% in 5 minutes, 60-70% in 10 minutes, 71-85% in 30 minutes with the mist spray made from preparation example 1-7, especially preparation example 1 and preparation example 7.

Test 4: the efficacy of preventing and curing virus and germs after using the mosquito repellent solution of preparation example 1-7

TABLE 4
the efficacy of preventing and curing virus and germs after using the mosquito
repellent solution of preparation example 1-7

| | Percentage of killing virus (%) | | | | | | | |
|---|---|---|---|---|---|---|---|---|
| | | | Infectious | Newcastle | Percentage of killing germs (%) | | | |
| | Influenza | Corona | bronchitis | disease | | | Dysentery | Tubercle |
| treatment | virus | virus | virus | virus | streptococcus | typhoaemia | bacillus | bacillus |
| Preparation | 52.3 | 47.9 | 53.2 | 56.6 | 75.1 | 78.9 | 80.1 | 76.9 |
| example 1 | | | | | | | | |
| Preparation | 42.1 | 32.1 | 38.7 | 30.2 | 62.3 | 66.3 | 57.2 | 54.6 |
| example 2 | | | | | | | | |
| Preparation | 40.0 | 34.5 | 28.7 | 36.7 | 50.3 | 65.4 | 54.5 | 55.3 |
| example 3 | | | | | | | | |
| Preparation | 35.8 | 40.1 | 34.6 | 38.7 | 54.2 | 61.2 | 52.0 | 60.3 |
| example 4 | | | | | | | | |
| Preparation | 33.2 | 31.3 | 37.5 | 44.3 | 55.4 | 60.7 | 60.2 | 65.1 |
| example 5 | | | | | | | | |
| Preparation | 34.1 | 28.7 | 30.4 | 42.1 | 56.1 | 58.6 | 52.1 | 51.4 |
| example 6 | | | | | | | | |
| Preparation | 48.9 | 48.7 | 51.4 | 58.7 | 74.6 | 77.2 | 77.2 | 77.1 |
| example 7 | | | | | | | | |

Table 4 showed that the mosquito repellent solution of preparation example 1-7 can kill virus as influenza virus, coronavirus, infectious bronchitis virus, Newcastle disease virus, also germs as streptococcus, typhoaemia, dysentery bacillus, and tubercle bacillus. The efficacy of preparation example 1 and 7 are better.

What is claimed is:

1. A mosquito repellent solution comprising:
0.1-9.9% organic acid of total weight as active ingredient, 88-99.6% water of total weight as solvent, the organic acid comprises at least one organic acid selected from the group consisting of acetic acid, propanoic acid, n-butyric acid, isobutyric acid, isobutyric acid, valeric acid, hexanoic acid, n-Heptanoic acid, L-Lactic acid and malic acid.

2. The mosquito repellent solution as recited in claim 1, wherein the organic acid is 1.0-8.0% by weight of the mosquito repellent solution, it is better that the organic acid is 2.0-6.0% by weight of the mosquito repellent solution.

3. The mosquito repellent solution as recited in claim 1, the water as solvent is distilled water, the distilled water has 90-98% by weight of the mosquito repellent solution, it is better that the distilled water has 93-96% by weight of the mosquito repellent solution.

4. The mosquito repellent solution as recited in claim 1, the organic acid is in a food level.

5. The mosquito repellent solution as recited in claim 1, wherein 0.1-0.5% sodium chloride or citric acid of total weight is used as antiseptic.

6. The mosquito repellent solution as recited in claim 1, wherein 0.1-0.5% water-soluble fruit flavor spices of total weight is added as sauce.

7. A preparation method for the mosquito repellent solution as recited in claim 1 comprising following steps:
place a right proportion of distilled water and organic acid in a heating mixer, heat the mixture of distilled water and organic acid to 15-35° C. while constantly stirring the mixture to promote dissolve, then yield the final product of the mosquito repellent solution.

8. The preparation method as recited in claim 7, wherein antiseptics and sauce are added in order during constantly stirring to promote dissolve.

9. The preparation method as recited in claim 7, wherein the mixture is heated to 20-30° C., better to 20-25° C.

10. A use of the mosquito repellent solution as recited in claim 1 comprising preventing mosquito bite, relieving itching, detumescence and air sterilization.
